(19) United States
(12) Patent Application Publication
(10) Pub. No.: US 2018/0151302 A1
(43) Pub. Date: May 31, 2018

(54) PHOTOELECTRIC CONVERSION ELEMENT AND METHOD FOR PRODUCING THE SAME

(71) Applicant: Sharp Kabushiki Kaisha, Sakai City (JP)

(72) Inventors: Hiroshi SUGIMURA, Sakai City (JP); Kohichi TORIYAMA, Sakai City (JP); Takahiro KURAUCHI, Sakai City (JP)

(21) Appl. No.: 15/790,096

(22) Filed: Oct. 23, 2017

(30) Foreign Application Priority Data
Nov. 30, 2016 (JP) 2016-233005

(51) Int. Cl. H01G 9/20; H01L 51/00; C07C 211/54; H01L 51/42; H01G 9/00

(52) U.S. Cl. CPC H01G 9/2018; H01L 51/0077; H01L 51/0059; H01L 2251/303; H01L 51/4253; H01L 51/0003; H01G 9/0029; C07C 211/54

(57) ABSTRACT

A photoelectric conversion element according to the present disclosure includes an electron-transporting layer, a hole-transporting layer, and a light-absorbing layer interposed between the electron-transporting layer and the hole-transporting layer. The hole-transporting layer contains a compound represented by Formula (1) below;

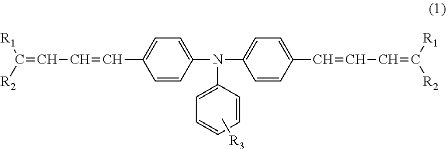

### BACKGROUND

### 1. Field

The present disclosure relates to a photoelectric conversion element and a method for producing the photoelectric conversion element.

### 2. Description of the Related Art

Photoelectric conversion elements are used in, for example, various photosensors, copying machines, and solar cells. In particular, the use of solar cells as a representative form of using renewable energy is spreading widely. Examples of such solar cells include silicon solar cells, CIGS solar cells, and CdTe solar cells.

Instead of the inorganic materials used in such solar cells, the use of organic materials as photoelectric conversion materials has been studied to develop organic thin-film solar cells and dye-sensitized solar cells. Since these solar cells can be produced by a coating process without using a vacuum process, they are possibly produced at low costs and thus expected to serve as next-generation solar cells.

However, such organic thin-film solar cells and dye-sensitized solar cells still have not only lower photoelectric conversion efficiency but also lower durability than solar cells in which inorganic materials are used.

In recent years, there has been proposed a solar cell including an electrolyte and a lead complex with a perovskite crystal structure as a photoelectric conversion material.

It has further been suggested that the electrolyte used for solar cells referred to above be replaced with 2,2′,7,7′-tetrakis(N,N-dimethoxyphenylamine)-9,9′-spirobifluorene (hereinafter abbreviated as spiro-OMeTAD), which is a solid organic hole-transporting material, and the conversion efficiency is found to exceed 10%.

Solid-state organic-inorganic hybrid solar cells (organic-inorganic hybrid photoelectric conversion elements) including a compound with a perovskite structure as a photoelectric conversion material have since been rapidly developed, and the conversion efficiency exceeds 20%. According to most reports, such organic-inorganic hybrid solar cells include spiro-OMeTAD with the following structure as a hole-transporting material.

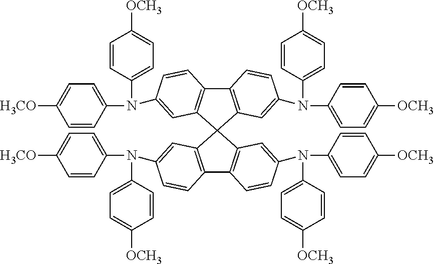 [SMILES: COc1ccc(N(c2ccc(OC)cc2)c2ccc3c(c2)C2(c4cc(N(c5ccc(OC)cc5)c5ccc(OC)cc5)ccc4-3)c3cc(N(c4ccc(OC)cc4)c4ccc(OC)cc4)ccc3-c3ccc(N(c4ccc(OC)cc4)c4ccc(OC)cc4)cc32)cc1]

Spiro-OMeTAD is a special material that has a low charge-transporting ability when in a neutral state and has a charge-transporting ability when in an oxidation state. In actual applications, for example, an oxidizing agent is added to a coating liquid containing spiro-OMeTAD, or the coating liquid is oxidized with air by exposing it to air for one day and one night before use. Spiro-OMeTAD is thus associated with handling difficulty during production and with difficulty in controlling the photoelectric conversion characteristics even after production.

In addition to the fluorene compound, attempts to use, for example, a triallylamine-based compound and a butadiene-based compound as hole-transporting materials have been made. The characteristics of these compounds were not superior to those of spiro-OMeTAD under present circumstances (see WO 2015/016107A1 and Chem. Commun., 2014, 50, 6931-6934).

### SUMMARY

In spite of promising organic-inorganic hybrid photoelectric conversion elements, spiro-OMeTAD used as a hole-transporting material is difficult to synthesize and expensive. For these reasons, application of such photoelectric conversion elements to large-scale solar cells leads to large production costs.

It is thus desirable to provide a photoelectric conversion element that has a high photoelectric conversion efficiency and high durability and which can be produced at low costs.

According to an aspect of the disclosure, there is provided a photoelectric conversion element including an electron-transporting layer, a hole-transporting layer, and a light-absorbing layer interposed between the electron-transporting layer and the hole-transporting layer. The hole-transporting layer contains a compound represented by Formula (1) below:

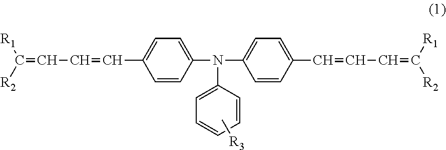

where R₁ represents a hydrogen atom, a substituted or unsubstituted alkyl group, or a substituted or unsubstituted aryl group, R₂ represents a hydrogen atom, a substituted or unsubstituted alkyl group, or a substituted or unsubstituted aryl group (excluding a case where R₁ and R₂ simultaneously represent a hydrogen atom), and R₃ represents a hydrogen atom, a substituted or unsubstituted alkyl group, a substituted or unsubstituted alkoxy group, a substituted or unsubstituted aryl group, or a substituted or unsubstituted aralkyl group.

### BRIEF DESCRIPTION OF THE DRAWINGS

FIG. 1 is a schematic sectional view of a photoelectric conversion element according to an embodiment of the disclosure; and

FIG. 2 illustrates a crystal lattice of a perovskite structure.

### DESCRIPTION OF THE EMBODIMENTS

A photoelectric conversion element according to the present disclosure includes an electron-transporting layer, a hole-transporting layer, and a light-absorbing layer interposed between the electron-transporting layer and the hole-transporting layer. The hole-transporting layer contains a compound represented by Formula (1) below:

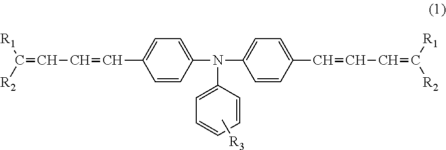

where R₁ represents a hydrogen atom, a substituted or unsubstituted alkyl group, or a substituted or unsubstituted aryl group, R₂ represents a hydrogen atom, a substituted or unsubstituted alkyl group, or a substituted or unsubstituted aryl group (excluding a case where R₁ and R₂ simultaneously represent a hydrogen atom), and R₃ represents a hydrogen atom, a substituted or unsubstituted alkyl group, a substituted or unsubstituted alkoxy group, a substituted or unsubstituted aryl group, or a substituted or unsubstituted aralkyl group.

The hole-transporting layer in the photoelectric conversion element of the present disclosure may contain a compound represented by Formula (1) where R₁ represents a hydrogen atom, a methyl group, an ethyl group, a phenyl group, a naphthyl group, or a tolyl group, R₂ represents a hydrogen atom, a methyl group, an ethyl group, a phenyl group, a naphthyl group, or a tolyl group, and R₃ represents a hydrogen atom, a methyl group, an ethyl group, a methoxy group, an ethoxy group, a phenyl group, a naphthyl group, a tolyl group, a benzyl group, or a phenethyl group. Because of this compound, the photoelectric conversion element of the present disclosure has a high photoelectric conversion efficiency.

The hole-transporting layer in the photoelectric conversion element of the present disclosure may contain a compound represented by Formula (1) where R₁ and R₂ each represent a p-tolyl group and R₃ represents a p-methoxy group. Because of this compound, the photoelectric conversion element of the present disclosure has a high photoelectric conversion efficiency.

The light-absorbing layer in the photoelectric conversion element of the present disclosure may contain a compound with an organic-inorganic hybrid perovskite structure. Because of this compound, the photoelectric conversion element of the present disclosure has a high photoelectric conversion efficiency.

The compound with an organic-inorganic hybrid perovskite structure may be a compound represented by Formula (2): CH₃NH₃PbX₃ where X represents a halogen atom. Because of this compound, the photoelectric conversion element of the present disclosure has a high photoelectric conversion efficiency.

The compound with an organic-inorganic hybrid perovskite structure may be a compound represented by Formula (3): CH₃NH₃PbI₃. Because of this compound, the photoelectric conversion element of the present disclosure has a high photoelectric conversion efficiency.

The electron-transporting layer in the photoelectric conversion element of the present disclosure may contain a compact titanium oxide layer and a porous titanium oxide layer on the compact titanium oxide layer. The light-absorbing layer may be disposed on the porous titanium oxide layer. This structure allows the light-absorbing layer and the hole-transporting layer to be formed in pores of the porous titanium oxide layer, can increase the area of contact between the porous titanium oxide layer and the light-absorbing layer and the area of contact between the light-absorbing layer and the hole-transporting layer, and allows electrons and holes produced by photoexcitation in the light-absorbing layer to efficiently undergo charge separation. As a result, the photoelectric conversion efficiency of the photoelectric conversion element can be improved. The formation of the compact titanium oxide layer can avoid contact between a first conductive layer and the hole-transporting layer and can suppress a decrease in photoelectromotive force.

According to an aspect of the disclosure, there is provided a method for producing a photoelectric conversion element. The method includes dissolving the compound represented by Formula (1) in an organic solvent to prepare a coating liquid; applying the coating liquid to a light-absorbing layer; and removing the organic solvent to form a hole-transporting layer. The photoelectric conversion element can be produced at low costs by forming the hole-transporting layer by a coating method.

Embodiments of the present disclosure will be described below with reference to the drawings. The structures provided in the drawings and the following description are illustrative only, and the scope of the present disclosure is not limited to embodiments described the drawings and the following description.

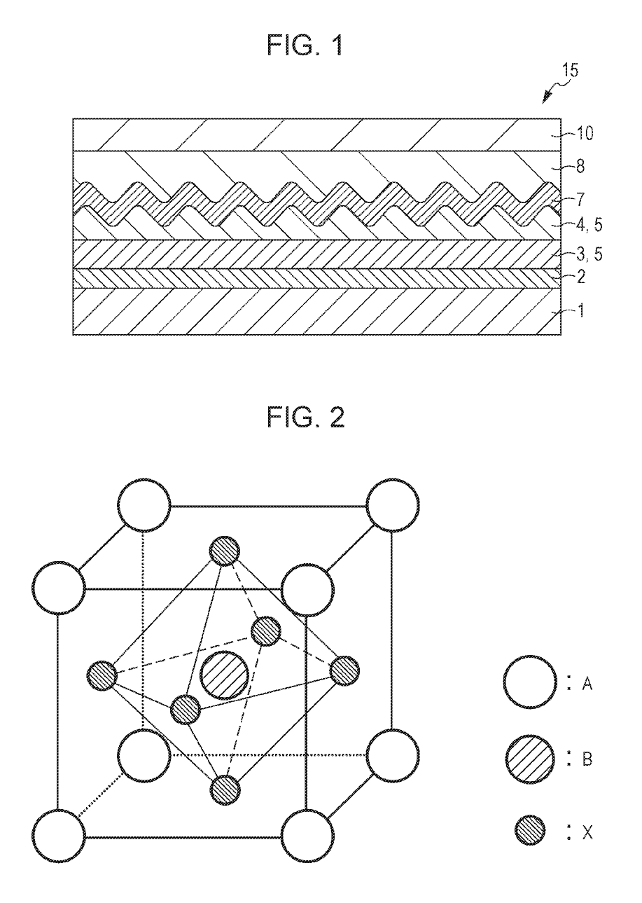

FIG. 1 is a schematic sectional view of a photoelectric conversion element according to this embodiment.

A photoelectric conversion element 15 according to this embodiment includes an electron-transporting layer 5, a hole-transporting layer 8, and a light-absorbing layer 7 interposed between the electron-transporting layer 5 and the hole-transporting layer 8. The hole-transporting layer 8 contains a compound represented by Formula (1) below:

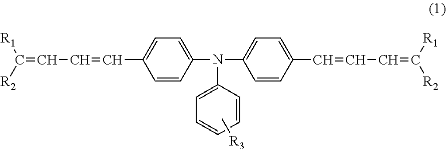

where R₁ represents a hydrogen atom, a substituted or unsubstituted alkyl group, or a substituted or unsubstituted aryl group, R₂ represents a hydrogen atom, a substituted or unsubstituted alkyl group, or a substituted or unsubstituted aryl group (excluding a case where R₁ and R₂ simultaneously represent a hydrogen atom), and R₃ represents a hydrogen atom, a substituted or unsubstituted alkyl group, a substituted or unsubstituted alkoxy group, a substituted or unsubstituted aryl group, or a substituted or unsubstituted aralkyl group.

The photoelectric conversion element 15 according to this embodiment can contain a first conductive layer 2 and a second conductive layer 10. In this case, the electron-transporting layer 5, the light-absorbing layer 7, the hole-transporting layer 8, and the second conductive layer 10 can be disposed in this order on the first conductive layer 2.

The electron-transporting layer 5 can have a compact titanium oxide layer 3 and a porous titanium oxide layer 4. In this case, the compact titanium oxide layer 3, the porous titanium oxide layer 4, the light-absorbing layer 7, the hole-transporting layer 8, and the second conductive layer 10 can be disposed in this order on the first conductive layer 2.

The photoelectric conversion element 15 according to this embodiment can contain a substrate 1. In this case, the first conductive layer 2, the electron-transporting layer 5, the light-absorbing layer 7, the hole-transporting layer 8, and the second conductive layer 10 can be disposed in this order on the substrate 1.

The photoelectric conversion element 15 according to this embodiment may be an organic-inorganic hybrid photoelectric conversion element.

The photoelectric conversion element according to this embodiment and a method for producing the photoelectric conversion element will be described below.

### 1. First Conductive Layer, Substrate

The first conductive layer 2 is a layer that functions as a cathode of the photoelectric conversion element 15. Examples of cathode materials that can be used for the first conductive layer 2 include conductive transparent materials, such as copper iodide (CuI), indium tin oxide (ITO), tin oxide (SnO₂), fluorine-doped tin oxide (FTO), aluminum-doped zinc oxide (AZO), indium zinc oxide (IZO), and gallium-doped zinc oxide (GZO); and metal sodium, a sodium-potassium alloy, metal lithium, metal magnesium, metal aluminum, a magnesium-silver mixture, a magnesium-indium mixture, an aluminum-lithium alloy, an aluminum-aluminum oxide (Al/Al₂O₃) mixture, and an aluminum-lithium fluoride (Al/LiF) mixture. These materials may be used alone or in combination of two or more.

The first conductive layer 2 can be produced, for example, by forming a film made of a cathode material on the substrate 1 by an ordinary method, such as vapor deposition. Examples of the substrate 1 include, but are not limited to, transparent glass substrates made of, for example, soda-lime glass and alkali-free glass, ceramic substrates, and transparent plastic substrates. The substrate 1 may be a commercial product.

In a case where light enters the photoelectric conversion element 15 from the substrate 1 side, the substrate 1 may be a transparent substrate, and the first conductive layer 2 may be a transparent electrode. In a case where light enters the photoelectric conversion element 15 from the second conductive layer 10 side, the second conductive layer 10 may be a transparent electrode. The first conductive layer 2 and the second conductive layer 10 may both be transparent electrodes.

The thickness of the first conductive layer 2 may be in the range from about 0.4 to about 1.5 μm. If the thickness of the first conductive layer 2 is less than about 0.4 μm, it is difficult to obtain sufficient conductivity. If the thickness of the first conductive layer 2 is more than about 1.5 μm, the first conductive layer 2 tends to have a low transmittance, which may result in a low photoelectric conversion efficiency.

### 2. Electron-Transporting Layer

The electron-transporting layer 5 is a layer that transports electrons produced by photoexcitation in the light-absorbing layer 7 to the first conductive layer 2. Therefore, the electron-transporting layer 5 is made of a material that allows electrons produced in the light-absorbing layer 7 to easily move to the electron-transporting layer 5 and allows the electrons in the electron-transporting layer 5 to easily move to the first conductive layer 2. For example, the electron-transporting layer 5 is a titanium oxide layer. The electron-transporting layer 5 may have a compact titanium oxide layer 3 and a porous titanium oxide layer 4. The compact titanium oxide layer 3 can be disposed on the first conductive layer 2, and the porous titanium oxide layer 4 can be disposed on the compact titanium oxide layer 3. In addition, the electron-transporting layer 5 may have a compact layer made of a material other than titanium oxide and a porous layer formed on the compact layer and made of a material other than titanium oxide.

The compact titanium oxide layer 3 together with the porous titanium oxide layer 4 forms the electron-transporting layer 5. The compact titanium oxide layer 3 avoids contact between the first conductive layer 2 and the second conductive layer 10. This contact may cause a decrease in electromotive force. The formation of the compact titanium oxide layer 3 can also avoid contact between the first conductive layer 2 and the hole-transporting layer 8. This can suppress a decrease in photoelectromotive force. In this embodiment, the term “compact layer” refers to a layer that has few pores and through which a solution or the like does not permeate during formation of the light-absorbing layer 7. The term “porous layer” refers to a layer that has large pores and through which a solution or the like permeates during formation of the light-absorbing layer 7. The porous layer has an effect of increasing the area of contact between the electron-transporting layer 5 and the light-absorbing layer 7.

The thickness of the compact titanium oxide layer 3 is preferably from about 5 to about 200 nm, and more preferably from about 10 to about 100 nm.

The compact titanium oxide layer 3 can be formed, for example, by preparing a coating liquid containing a titanium chelate compound, and applying the coating liquid to the first conductive layer 2 by a film forming method, such as spin coating, screen printing, spray pyrolysis, or aerosol deposition, followed by firing. After firing, the compact titanium oxide layer 3 may be immersed in an aqueous solution of titanium tetrachloride. This process can increase the compactness of the compact titanium oxide layer 3.

The titanium chelate compound that can be used for forming the compact titanium oxide layer 3 may be a commercial product, such as TYZOR (registered trademark) AA series available from DuPont. The titanium chelate compound may be a compound with an acetoacetate chelate group or a compound with a β-diketone chelate group.

Examples of compounds with an acetoacetate chelate group, among titanium chelate compounds that can be used for forming the compact titanium oxide layer 3, include, but are not limited to, diisopropoxy titanium bis(methylacetoacetate), diisopropoxy titanium bis(ethylacetoacetate), diisopropoxy titanium bis(propylacetoacetate), diisopropoxy titanium bis (butylacetoacetate), dibutoxy titanium bis(methylacetoacetate), dibutoxy titanium bis(ethylacetoacetate), triisopropoxy titanium (methylacetoacetate), triisopropoxy titanium (ethylacetoacetate), tributhoxy titanium (methylacetoacetate), tributhoxy titanium (ethylacetoacetate), isopropoxy titanium tri(methylacetoacetate), isopropoxy titanium tri(ethylacetoacetate), isobutoxy titanium tri(methylacetoacetate), and isobutoxy titanium tri(ethylacetoacetate). Examples of compounds with a β-diketone chelate group include, but are not limited to, diisopropoxy titanium bis(acetylacetonate), diisopropoxy titanium bis(2,4-heptanedionate), dibutoxy titanium bis(acetylacetonate), dibutoxy titanium bis(2,4-heptanedionate), triisopropoxy titanium (acetylacetonate), triisopropoxy titanium (2,4-heptanedionate), tributhoxy titanium (acetylacetonate), tributhoxy titanium (2,4-heptanedionate), isopropoxy titanium tri(acetylacetonate), isopropoxy titanium tri(2,4-heptanedionate), isobutoxy titanium tri(acetylacetonate), and isobutoxy titanium tri(2,4-heptanedionate).

The porous titanium oxide layer 4 together with the compact titanium oxide layer 3 forms the electron-transporting layer 5. The light-absorbing layer 7 and the hole-transporting layer 8 are formed in pores of the porous titanium oxide layer 4. The formation of the light-absorbing layer 7 in pores can increase the area of contact between the porous titanium oxide layer 4 and the light-absorbing layer 7 and thus allows electrons produced by photoexcitation in the light-absorbing layer 7 to efficiently move to the porous titanium oxide layer 4. The formation of the hole-transporting layer 8 in pores can increase the area of contact between the light-absorbing layer 7 and the hole-transporting layer 8 and thus allows holes produced by photoexcitation in the light-absorbing layer 7 to efficiently move to the hole-transporting layer 8. Therefore, the electrons and holes produced by photoexcitation in the light-absorbing layer 7 can efficiently undergo charge separation, which can suppress recombination of the electrons and holes produced by photoexcitation. As a result, the photoelectric conversion efficiency of the photoelectric conversion element 15 can be improved.

The thickness of the porous titanium oxide layer 4 is preferably from 100 to 20000 nm, and more preferably from 200 to 1500 nm.

The porous titanium oxide layer 4 can be formed, for example, by preparing a coating liquid containing titanium oxide particles, and applying the coating liquid to the compact titanium oxide layer 3 by a film forming method, such as spin coating, screen printing, spray pyrolysis, or aerosol deposition, followed by firing. When the coating liquid contains an organic binder, the organic binder is removed by a firing process.

Titanium oxide has several crystal forms. Titanium oxide may be of anatase type in order to form the porous titanium oxide layer 4.

The coating liquid used to form the porous titanium oxide layer 4 can be prepared by, for example, dispersing titanium oxide particles (e.g., P-25 available from Nippon Aerosil Co., Ltd.) in an alcohol (e.g., ethanol) or diluting a titanium oxide paste (e.g., PST-18NR available from JGC Catalysts and Chemicals Ltd.) with an alcohol (e.g., ethanol).

Examples of the organic binder that can be used for the coating liquid include, but are not limited to, ethyl cellulose and acrylic resins. Acrylic resins may be used because they can easily be decomposed at low temperature and lead to small amounts of organic residues even in the case of low-temperature firing. Acrylic resins that are decomposable at a temperature as low as about 300° C. may be used. Examples of such acrylic resins include polymers produced by polymerization of at least one (meth)acrylic monomer selected from the group consisting of methyl (meth)acrylate, ethyl (meth)acrylate, propyl (meth)acrylate, n-butyl (meth)acrylate, tert-butyl (meth)acrylate, isobutyl (meth)acrylate, cyclohexyl (meth) acrylate, 2-ethylhexyl (meth) acrylate, isoboronyl (meth)acrylate, n-stearyl (meth)acrylate, benzyl (meth)acrylate, and a (meth)acrylic monomer with a polyoxyalkylene structure.

The pore diameter of the porous titanium oxide layer 4 can be controlled by changing the particle size of titanium oxide particles or changing the type and amount of the organic binder added.

### 3. Light-Absorbing Layer

The light-absorbing layer 7 is a layer that absorbs light incident on the photoelectric conversion element 15 to produce electrons and holes. Specifically, in the light-absorbing layer 7, lower-energy electrons of a substance contained in the light-absorbing layer 7 are photoexcited by incident light to produce higher-energy electrons and holes. The electrons move to the electron-transporting layer 5 and the holes move to the hole-transporting layer, such that the electrons and the holes undergo charge separation.

The light-absorbing layer 7 can contain an organic-inorganic hybrid compound. This compound is photoexcited to produce electrons and holes in the light-absorbing layer 7. Since organic-inorganic hybrid compounds have properties derived from inorganic materials, a photoelectric conversion layer containing such a compound is more durable than a photoelectric conversion layer composed of organic materials. The light-absorbing layer 7 may contain a compound with an organic-inorganic hybrid perovskite structure. The light-absorbing layer 7 may be a layer formed of a compound with an organic-inorganic hybrid perovskite structure.

The unit cell of the perovskite-type crystal structure is illustrated in FIG. 2. As illustrated in this figure, the compound with an organic-inorganic hybrid perovskite structure that can be used for the light-absorbing layer 7 has a cubic unit cell. This structure has an organic group A at each corner of the cube, a metal B at the center of the cube, and a halogen X at each face of the metal B-centered cube. This structure is represented by a general formula of A-B—X₃.

Specific examples of the organic group A in the general formula of A-B—X₃ include methylamine, ethylamine, propylamine, butylamine, pentylamine, hexylamine, dimethylamine, diethylamine, dipropylamine, dibutylamine, dipentylamine, dihexylamine, trimethylamine, triethylamine, tripropylamine, tributylamine, tripentylamine, trihexylamine, ethylmethylamine, methylpropylamine, butylmethylamine, methylpentylamine, hexylmethylamine, ethylpropylamine, ethylbutylamine, imidazole, azole, pyrrole, aziridine, azirine, azetidine, azete, azole, imidazoline, carbazole, and ions thereof (e.g., methylammonium (CH₃NH₃)), and phenethylammonium. Among these, methylamine, ethylamine, propylamine, butylamine, pentylamine, hexylamine, and ions thereof, and phenethylammonium are preferred. Methylamine, ethylamine, propylamine, and ions thereof (e.g., methylammonium (CH₃NH₃)) are more preferred.

Specific examples of the metal B in the general formula of A-B—X₃ include lead, tin, zinc, titanium, antimony, bismuth, nickel, iron, cobalt, silver, copper, gallium, germanium, magnesium, calcium, indium, aluminum, manganese, chromium, molybdenum, and europium. When the metal B is lead among them, the light-absorbing layer 7 has good properties. These elements may be used alone or in combination of two or more.

In the general formula of A-B—X₃, the halogen X is chlorine, bromine, or iodine. These elements may be used alone or in combination of two or more. At least one of X's may be iodine among these elements because the energy band gap can be narrowed.

The light-absorbing layer 7 may be a layer formed of a compound with a perovskite structure represented by CH₃NH₃PbX₃ where X represents a halogen atom. In the compound represented by CH₃NH₃PbX₃, X may represent an iodine atom.

The compound with a perovskite structure that can be used for the light-absorbing layer 7 can be synthesized by using as materials a compound represented by AX and a compound represented by BX₂. Specifically, the compound with a perovskite structure can be synthesized by mixing a solution of AX and a solution of BX₂ and heating the mixture under stirring (one-step process). Alternatively, the compound with a perovskite structure can be synthesized by applying a solution of BX₂ to, for example, the porous titanium oxide layer 4 to form a coating film, and applying a solution of AX to the coating film so that BX₂ and AX react with each other (two-step process). Either the one-step process or the two-step process can be used to form the light-absorbing layer 7. Examples of the application method include, but are not limited to, spin coating, screen printing, and dip coating.

### 4. Hole-Transporting Layer

The hole-transporting layer 8 is a layer that captures holes produced in the light-absorbing layer 7 and allows the holes to move to the second conductive layer 10 which is an anode. The hole-transporting layer 8 contains a compound represented by Formula (1) below:

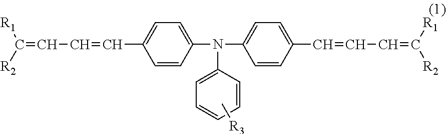

where R₁ represents a hydrogen atom, a substituted or unsubstituted alkyl group, or a substituted or unsubstituted aryl group, R₂ represents a hydrogen atom, a substituted or unsubstituted alkyl group, or a substituted or unsubstituted aryl group (excluding a case where R₁ and R₂ simultaneously represent a hydrogen atom), and R₃ represents a hydrogen atom, a substituted or unsubstituted alkyl group, a substituted or unsubstituted alkoxy group, a substituted or unsubstituted aryl group, or a substituted or unsubstituted aralkyl group. The hole-transporting layer 8 may be a layer composed of this compound. The compound represented by Formula (1) is used as a hole-transporting material to achieve high photoelectric conversion efficiency.

Examples of the substituted or unsubstituted alkyl group include a methyl group (Me) and an ethyl group (Et). Examples of the substituted or unsubstituted alkoxy group include a methoxy group (OMe) and an ethoxy group (OEt). Examples of the substituted or unsubstituted aryl group include a phenyl group, a naphthyl group, and a tolyl group. Examples of the substituted or unsubstituted aralkyl group include a benzyl group and a phenethyl group.

Specific examples 1 to 20 of R₁, R₂, and R₃ in Formula (1) are illustrated in Table 1. The hole-transporting material in the hole-transporting layer 8 is not limited to compounds represented by Formula (1) containing R₁, R₂, and R₃ in Numbers 1 to 20 illustrated in Table 1.

The hole-transporting material in the hole-transporting layer 8 may be a compound represented by Formula (1) where R₁ and R₂ each represent a p-tolyl group and R₃ represents a p-methoxy group (a compound represented by Formula (1) containing R₁, R₂, and R₃ in Number 13 illustrated in Table 1).

TABLE 1

| No. | R₁ | R₂ | R₃ |
|---|---|---|---|
| 1 | 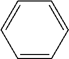 | 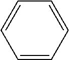 | H |
| 2 | 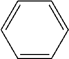 | 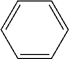 | p-CH₃ |
| 3 | 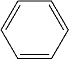 | 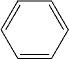 | p-OCH₃ |
| 4 | 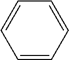 | 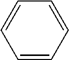 | p-C₂H₅ |
| 5 | 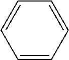 | 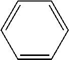 | p- OC₂H₅ |
| 6 | 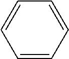 | H | H |
| 7 | 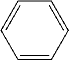 | H | p-CH₃ |
| 8 | 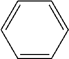 | H | p-OCH₃ |
| 9 | 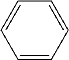 | H | p-C₂H₅ |
| 10 | 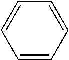 | H | p- OC₂H₅ |
| 11 | 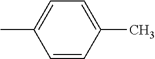 | 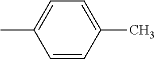 | H |
| 12 | 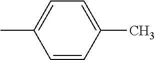 | 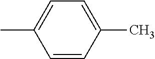 | p-CH₃ |
| 13 | 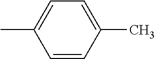 | 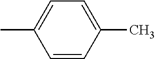 | p-OCH₃ |
| 14 | 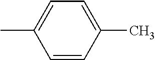 | 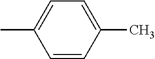 | p-C₂H₅ |
| 15 | 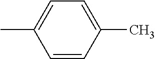 | 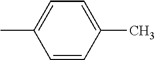 | p- OC₂H₅ |
| 16 | 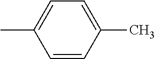 | H | H |
| 17 | 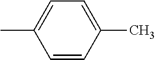 | H | p-CH₃ |
| 18 | 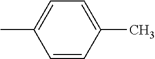 | H | p-OCH₃ |
| 19 | 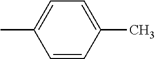 | H | p-C₂H₅ |
| 20 | 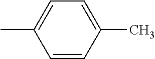 | H | p- OC₂H₅ |

The compound represented by Formula (1) can be synthesized by, for example, the method described in Japanese Patent No. 3181799. A commercially available compound represented by Formula (1) can also be used.

The hole-transporting layer 8 can be formed by dissolving the compound represented by Formula (1) in an organic solvent to prepare a coating liquid, applying the coating liquid to the light-absorbing layer 7, and then removing the organic solvent.

Since the organic solvent is applied to the light-absorbing layer 7, the organic solvent may be a solvent that does not damage the crystal structure of the organic-inorganic hybrid compound. Specific examples of the organic solvent include chlorobenzene and toluene.

Examples of the application method include, but are not limited to, spin coating, screen printing, and dip coating. Since the method for producing the photoelectric conversion element 15 in this embodiment includes such a step of forming the hole-transporting layer 8, an organic-inorganic hybrid photoelectric conversion element having high photoelectric conversion efficiency and high durability can easily be produced and can be used to produce a large-scale solar power generation system. Since inexpensive materials can be used in the method for producing the photoelectric conversion element 15 in this embodiment, production costs can be reduced.

The thickness of the hole-transporting layer 8 is preferably from about 20 to about 500 nm, and more preferably from about 50 to about 150 nm. The hole-transporting layer 8 may be an amorphous layer. Although the compound represented by Formula (1) is difficult to crystallize, the hole-transporting layer 8 may contain, for example, an organic binder resin and a plasticizer in order to more assuredly reduce crystallization of the compound represented by Formula (1).

### 5. Second Conductive Layer

The second conductive layer 10 is a layer that functions as an anode of the photoelectric conversion element 15. Anode materials that can be used for the second conductive layer 10 are not limited, and known materials may be used. Examples of anode materials include metals, such as gold, silver, and platinum; conductive transparent materials, such as copper iodide (CuI), indium tin oxide (ITO), tin oxide (SnO₂), fluorine-doped tin oxide (FTO), aluminum-doped zinc oxide (AZO), indium zinc oxide (IZO), and gallium-doped zinc oxide (GZO); and conductive transparent polymers. These materials may be used alone or in combination of two or more.

The second conductive layer 10 can be produced, for example, by forming a film made of an anode material on the hole-transporting layer 8 by an ordinary method, such as vapor deposition. In the case of a gold electrode, the thickness of the second conductive layer 10 may be from about 50 nm to about 100 nm. In a case where light enters the photoelectric conversion element 15 from the second conductive layer 10 side, the second conductive layer 10 may be a transparent electrode.

### Production of Photoelectric Conversion Element

Photoelectric conversion elements of Examples 1 to 4 and Comparative Examples 1 and 2 were produced.

### EXAMPLE 1

### 1. First Conductive Layer

A glass substrate (thickness 2.2 mm) on which a fluorine-doped SnO₂ conductive film (FTO-deposited film) was formed by vapor deposition was prepared and cut into pieces measuring 25 mm×25 mm. The obtained pieces were subjected to ultrasonic cleaning for 1 hour and then irradiated with UV light for 30 minutes.

### 2. Compact Titanium Oxide Layer (Electron-Transporting Layer)

First, a 75% (by mass) solution of titanium(IV) bis(acetylacetonate) diisopropoxide in 1-butanol (available from Sigma-Aldrich) was diluted with 1-butanol to prepare a compact titanium oxide layer solution (coating liquid) containing 0.02 mol/L of a titanium chelate compound. Next, the compact titanium oxide layer solution was applied to the FTO-deposited film of the glass substrate, which had been irradiated with UV light in “1. First Conductive Layer”, by spin coating. The obtained coating layer was heated at 450° C. for 15 minutes to form a compact titanium oxide layer having a thickness of 50 nm.

### 3. Porous Titanium Oxide Layer (Electron-Transporting Layer)

First, 1 g of a dispersion of titanium oxide in ethanol (PST-18NR available from JGC Catalysts and Chemicals Ltd.) was diluted with 2.5 g of ethanol to prepare a coating liquid for a porous titanium oxide layer. Next, the coating liquid for a porous titanium oxide layer was applied by spin coating to the compact titanium oxide layer formed in “2. Compact Titanium Oxide Layer” and fired to form a porous titanium oxide layer having a thickness of 300 nm. The firing was performed at 450° C. for 1 hour.

### 4. Light-Absorbing Layer

First, 1.4 g of PbI₂(available from Tokyo Chemical Industry Co., Ltd.) was dissolved in 3 ml of N,N-dimethylformamide (DMF) under heating to prepare a coating liquid. This coating liquid was applied by spin coating to the porous titanium oxide layer formed in “3. Porous Titanium Oxide Layer” to form a coating film. The coating film was yellow, which was the same color as that of the coating liquid.

Next, 0.4 g of CH₃NH₃I (available from Wako Pure Chemical Industries, Ltd.) was dissolved in 40 ml of isopropyl alcohol, and the resulting solution was placed in a beaker. A glass plate having a coating film of PbI₂ formed in advance was immersed in the solution of CH₃NH₃I. The color of the coating film immediately changed from yellow to black, indicating that a compound with a perovskite-type crystal structure represented by CH₃NH₃PbI₃ was formed. The light-absorbing layer was formed accordingly by the two-step process.

### 5. Hole-Transporting Layer

First, 85 mg of the compound (hole-transporting material) represented by Formula (1) having R₁, R₂, and R₃ in Number 1 illustrated in Table 1 was dissolved in 2 ml of toluene to prepare a hole-transporting layer solution (coating liquid). This solution was applied by spin coating to the light-absorbing layer formed in “4. Light-Absorbing Layer”, and the coating film was next dried by heating to remove the organic solvent, forming a hole-transporting layer having a thickness of 100 nm.

### 6. Second Conductive Layer

On the hole-transporting layer formed in “5. Hole-Transporting Layer”, a gold-deposited film with an area of 5 mm×5 mm and a thickness of 80 nm was formed as an anode by vacuum deposition, producing an organic-inorganic hybrid photoelectric conversion element of Example 1.

### EXAMPLE 2

An organic-inorganic hybrid photoelectric conversion element of Example 2 was produced by the same method as that in Example 1 except that a hole-transporting layer was formed by using a compound represented by Formula (1) having R₁, R₂, and R₃ in Number 10 illustrated in Table 1 instead of the hole-transporting material used in Example 1.

### EXAMPLE 3

An organic-inorganic hybrid photoelectric conversion element of Example 3 was produced by the same method as that in Example 1 except that a hole-transporting layer was formed by using a compound represented by Formula (1) having R₁, R₂, and R₃ in Number 13 illustrated in Table 1 instead of the hole-transporting material used in Example 1.

### EXAMPLE 4

An organic-inorganic hybrid photoelectric conversion element of Example 4 was produced by the same method as that in Example 1 except that a hole-transporting layer was formed by using a compound represented by Formula (1) having R₁, R₂, and R₃ in Number 20 illustrated in Table 1 instead of the hole-transporting material used in Example 1.

### COMPARATIVE EXAMPLE 1

An organic-inorganic hybrid photoelectric conversion element of Comparative Example 1 was produced by the same method as that in Example 1 except that a hole-transporting layer was formed in the following manner.

A coating liquid for a hole-transporting layer was prepared by dissolving 144 mg of spiro-OMeTAD (available from Luminescence Technology Corp.) serving as a hole-transporting material, 18 mg of lithium bis(trifluoromethanesulfonyl)imide (available from Kishida Chemical Co.,Ltd.) serving as an ion conductive agent, and 53 mg of 4-tert-butylpyridine (available from Tokyo Chemical Industry Co., Ltd.) in 2 ml of chlorobenzene (available from Kishida Chemical Co., Ltd.). To oxidize spiro-OMeTAD, the coating liquid for a hole-transporting layer was exposed to air for one day. Subsequently, the coating liquid for a hole-transporting layer was applied to the light-absorbing layer by spin coating. The obtained coating film was next dried by heating to remove the organic solvent, forming a hole-transporting layer having a thickness of 100 nm.

### COMPARATIVE EXAMPLE 2

An organic-inorganic hybrid photoelectric conversion element of Comparative Example 2 was produced by the same method as that in Example 1 except that a hole-transporting layer was formed by using, instead of the hole-transporting material used in Example 1, a compound represented by Formula (4) below:

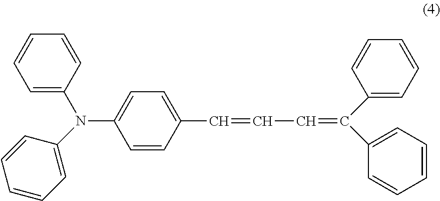 [SMILES: C(=Cc1ccc(N(c2ccccc2)c2ccccc2)cc1)C=C(c1ccccc1)c1ccccc1]

### Measurement of Photoelectric Conversion Efficiency

The produced organic-inorganic hybrid photoelectric conversion elements of Examples 1 to 4 and Comparative Examples 1 and 2 were measured for photoelectric conversion efficiency. Specifically, the I-V characteristics of the produced photoelectric conversion elements in which the anode was a gold-deposited film and the cathode was an FTO-deposited film were evaluated by irradiating the produced photoelectric conversion elements with simulated sunlight at 100 mW/cm² through an AM filter (AM-1.5) using a solar simulator (available from WACOM Electric Co., Ltd.). The measurement results are illustrated in Table 2.

TABLE 2

| | Short-Circuit | | | |
|---|---|---|---|---|
| | Current | Open-Circuit | | |
| | Jsc | Voltage | Fill Factor | Conversion |
| | mA/cm² | Voc V | FF | Efficiency η % |
| Example 1 | 13.2 | 0.95 | 0.62 | 7.8 |
| Example 2 | 10.1 | 0.85 | 0.58 | 5.0 |
| Example 3 | 14.3 | 0.94 | 0.65 | 8.7 |
| Example 4 | 11.5 | 0.82 | 0.61 | 5.8 |
| Comparative | 9.5 | 0.77 | 0.55 | 4.0 |
| Example 1 | | | | |
| Comparative | 5.9 | 0.5 | 0.66 | 1.9 |
| Example 2 | | | | |

The photoelectric conversion efficiency of the photoelectric conversion elements of Comparative Examples 1 and 2 was equal to or less than 4%, while the photoelectric conversion efficiency of the photoelectric conversion elements of Comparative Examples 1 to 4 was equal to or more than 5%. Therefore, the photoelectric conversion elements of the present disclosure having a hole-transporting layer containing the compound represented by Formula (1) as a hole-transporting material are found to have a photoelectric conversion efficiency higher than that of the photoelectric conversion elements containing a conventional hole-transporting material.

The photoelectric conversion element of the present disclosure can be used for solar cells. Since the present disclosure can provide an organic-inorganic hybrid photoelectric conversion element that can be easily produced and has high photoelectric conversion efficiency and high durability, the present disclosure can be applied to large-scale solar power generation systems (large-scale solar power plants) and power sources for portable devices.

The present disclosure contains subject matter related to that disclosed in Japanese Priority Patent Application JP 2016-233005 filed in the Japan Patent Office on Nov. 30, 2016, the entire contents of which are hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A photoelectric conversion element, comprising:
an electron-transporting layer;
a hole-transporting layer; and
a light-absorbing layer interposed between the electron-transporting layer and the hole-transporting layer,
wherein the hole-transporting layer contains a compound represented by Formula (1) below: 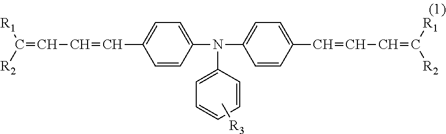
where R₁ represents a hydrogen atom, a substituted or unsubstituted alkyl group, or a substituted or unsubstituted aryl group, R₂ represents a hydrogen atom, a substituted or unsubstituted alkyl group, or a substituted or unsubstituted aryl group (excluding a case where R₁ and R₂ simultaneously represent a hydrogen atom), and R₃ represents a hydrogen atom, a substituted or unsubstituted alkyl group, a substituted or unsubstituted alkoxy group, a substituted or unsubstituted aryl group, or a substituted or unsubstituted aralkyl group.

2. The photoelectric conversion element according to claim 1, wherein the hole-transporting layer contains the compound represented by Formula (1) where R₁ represents a hydrogen atom, a methyl group, an ethyl group, a phenyl group, a naphthyl group, or a tolyl group, R₂ represents a hydrogen atom, a methyl group, an ethyl group, a phenyl group, a naphthyl group, or a tolyl group, and R₃ represents a hydrogen atom, a methyl group, an ethyl group, a methoxy group, an ethoxy group, a phenyl group, a naphthyl group, a tolyl group, a benzyl group, or a phenethyl group.

3. The photoelectric conversion element according to claim 1, wherein the hole-transporting layer contains the compound represented by Formula (1) where R₁ and R₂ each represent a p-tolyl group and R₃ represents a p-methoxy group.

4. The photoelectric conversion element according to claim 1, wherein the light-absorbing layer contains a compound with an organic-inorganic hybrid perovskite structure.

5. The photoelectric conversion element according to claim 4, wherein the compound with an organic-inorganic hybrid perovskite structure is a compound represented by Formula (2): CH₃NH₃PbX₃ where X represents a halogen atom.

6. The photoelectric conversion element according to claim 5, wherein the compound with an organic-inorganic hybrid perovskite structure is a compound represented by Formula (3): CH₃NH₃PbI₃.

7. The photoelectric conversion element according to claim 1, wherein the electron-transporting layer contains a compact titanium oxide layer and a porous titanium oxide layer on the compact titanium oxide layer, and the light-absorbing layer is disposed on the porous titanium oxide layer.

8. A method for producing the photoelectric conversion element according to claim 1, comprising:
dissolving the compound represented by Formula (1) in an organic solvent to prepare a coating liquid;
applying the coating liquid to the light-absorbing layer; and
removing the organic solvent to form the hole-transporting layer.
